(19) United States
(12) Patent Application Publication
(10) Pub. No.: US 2006/0067959 A1
(43) Pub. Date: Mar. 30, 2006

(54) Compositions and methods for treatment of solar damage

(76) Inventors: Marcel Nimni, Santa Monica, CA (US); Bo Han, Temple City, CA (US); Bruce J. Sand, Westlake Village, CA (US)

Correspondence Address: CHRISTIE, PARKER & HALE, LLP, PO BOX 7068, PASADENA, CA, 91109-7068, US

(21) Appl. No.: 10/951,824

(22) Filed: Sep. 28, 2004

(51) Int. Cl. A61K 8/49; A61K 31/375; A61K 31/198

(52) U.S. Cl. 424/401; 514/440; 514/474; 514/561

(57) ABSTRACT

An improved composition for treatment of the skin comprises: (1) methionine and cysteine in a quantity sufficient to accelerate restoration of the integrity and fullness of the skin; (2) a mixture of essential amino acids in a quantity sufficient to accelerate restoration of the integrity and fullness of the skin, the mixture comprising isoleucine, leucine, lysine, phenylalanine, threonine, tryptophan, valine, histidine, and arginine; (3) at least one antioxidant in a quantity sufficient to accelerate restoration of the integrity and fullness of the skin; (4) at least one cross-linking agent in a quantity sufficient to accelerate restoration of the integrity and fullness of the skin; (5) at least one metallic catalyst in a quantity sufficient to accelerate restoration of the integrity and fullness of the skin; (6) at least one transepidermal delivery agent in a quantity sufficient to promote delivery of the composition to the skin; and (7) a topical pharmaceutically acceptable carrier. The antioxidant can be ascorbic acid. The cross-linking agent can be proanthocyanidin or silybin. The metallic catalyst can be Cu (II) in a suitable cupric salt. The transepidermal delivery agent can be benzyl alcohol.

### BACKGROUND OF THE INVENTION

This invention is directed to compositions and methods for treating solar damage of the skin. This invention is particularly directed to compositions and methods for treating solar damage of the skin that restore cross-links in the collagen of the skin, and replenish the ground substance.

In skin trauma, including solar damage, trauma induced by incisions or wounds, or other conditions, the structural integrity of the skin, which is a manifestation of the support provided by the underlying dermis, is altered. The collagenous matrix in the dermis is damaged and replaced by elastin, which plays no role in repair or support. This condition is called elastosis and it may culminate in the appearance of rhytides (wrinkles) and loss of elasticity of the skin. Solar damage is a prime example of this process.

Repair or reversal of this damage requires collagen biosynthesis. This is true whether the trauma is incisional or merely solar-induced. The work of Kligman, Griffiths, and others has established that all trans-retinoic acid (tretinoin) can repair photoaged skin. This work has provided a focus to examine the more abundant dermal collagens as the key participants in photoaging. Collagen formation is reduced in photodamaged skin and topical tretinoin partially restores it.

Collagen biosynthesis alone, will not remedy this condition. Maturation of the newly generated collagen must take place before the tissue can be restored to its pre-trauma appearance. This is frequently overlooked or misunderstood in offering treatment options for solar-damaged skin or skin that has been subjected to incisional trauma.

Additionally, the role of the ground substance of the skin has not been properly appreciated. This ground substance was previously presumed to be biologically structured and inert. However, we now know that this ground substance is molecularly and structurally diverse, highly organized, and biologically active. The role of the ground substance is also frequently overlooked in offering treatment options for solar-damaged skin or skin that has been subjected to incisional trauma.

Therefore, there is a need for more effective treatment options for solar-damaged skin or skin that has been subjected to incisional trauma. In particular, there is a need for treatment options that address both collagen biosynthesis and collagen maturation. There is also a need for treatment options that address the role of the ground substance and the interaction of the ground substance with collagen.

### SUMMARY OF THE INVENTION

One aspect of the present invention is a composition for treatment of the skin comprising:

(1) methionine and cysteine in a quantity sufficient to accelerate restoration of the integrity and fullness of the skin;

(2) a mixture of essential amino acids in a quantity sufficient to accelerate restoration of the integrity and fullness of the skin, the mixture comprising isoleucine, leucine, lysine, phenylalanine, threonine, tryptophan, valine, histidine, and arginine;

(3) at least one antioxidant in a quantity sufficient to accelerate restoration of the integrity and fullness of the skin;

(4) at least one cross-linking agent in a quantity sufficient to accelerate restoration of the integrity and fullness of the skin;

(5) at least one metallic catalyst in a quantity sufficient to accelerate restoration of the integrity and fullness of the skin;

(6) at least one transepidermal delivery agent in a quantity sufficient to promote delivery of the composition to the skin; and

(7) a topical pharmaceutically acceptable carrier.

Typically, methionine is present in the composition at a concentration of from about 0.0005% to about 0.02% by weight. Preferably, methionine is present in the composition at a concentration of about 0.01% by weight.

Typically, cysteine is present in the composition at a concentration of from about 0.01% to about 0.4% by weight. Preferably, cysteine is present in the composition at a concentration of about 0.2% by weight.

Typically, the mixture of essential amino acids comprises from about 0.005% (w/w) to about 0.5% (w/w) of the composition. Preferably, the mixture of essential amino acids comprises from about 0.1% (w/w) to about 0.4% (w/w) of the composition. More preferably, the mixture of essential amino acids comprises about 0.3% (w/w) of the composition.

The at least one antioxidant is typically selected from the group consisting of lipoic acid or a lipoic acid derivative or analogue and ascorbic acid or a derivative of ascorbic acid. When the antioxidant is ascorbic acid or a derivative of ascorbic acid, it is typically a long-chain fatty acid ester of ascorbic acid selected from the group consisting of ascorbyl palmitate, ascorbyl myristate, and ascorbyl stearate. Preferably, the antioxidant is ascorbyl palmitate. Typically, the ascorbyl palmitate is present in the composition at a concentration of from about 0.1% (w/w) to about 0.6% (w/w). Preferably, the ascorbyl palmitate is present in the composition at a concentration of about 0.3% (w/w).

Alternatively, the antioxidant can be a constitutent of ginkgo or an isoflavone.

Typically, the metallic catalyst is copper in either its cuprous or cupric ionic form. Preferably, the copper is in cupric form. Typically, the copper is in the form of a copper salt selected from the group consisting of cupric acetate, cuprous acetate, cuprous chloride, cupric chloride, cuprous sulfate, and cupric sulfate. Preferably, the copper salt is selected from the group consisting of cupric acetate, cupric chloride, and cupric sulfate. More preferably, the copper salt is cupric chloride. Typically, the copper salt is present in the composition at a concentration of from about 1.0% to about 5.0% by weight. Preferably, the copper salt is present in the composition at a concentration of from about 1.5% to about 2.5% by weight. More preferably, the copper salt is present in the composition at a concentration of about 2.0% by weight.

Typically, the mixture of essential amino acids (not including cysteine or methionine) comprises:

(a) from about 5% to about 20% of isoleucine;

(b) from about 5% to about 20% of leucine;

(c) from about 10% to about 25% of lysine;

(d) from about 5% to about 20% of phenylalanine;

(e) from about 5% to about 25% of threonine;

(f) from about 5% to about 20% of tryptophan;

(g) from about 10% to about 25% of valine;

(h) from about 5% to about 20% of histidine; and

(i) from about 5% to about 20% of arginine.

Preferably, the mixture of essential amino acids (not including cysteine or methionine) comprises:

(a) about 8.48% of isoleucine;

(b) about 11.29% of leucine;

(c) about 14.68% of lysine;

(d) about 8.48% of phenylalanine;

(e) about 12.43% of threonine;

(f) about 7.91% of tryptophan;

(g) about 16.94% of valine;

(h) about 8.48% of histidine; and

(i) about 11.29% of arginine.

Typically, the crosslinking agent is a bioflavonoid. Preferably, the bioflavonoid is selected from the group consisting of quercetin, quercitrin, kaempferol, kaempferol 3-rutinoside, 3′-methoxy kaempferol 3-rutinoside, 5,8,4′-trihydroxyl-6,7-dimethoxyflavone, catechin, epicachetin, epicachetin gallate, epigallocachetin gallate, hesperidin, naringin, rutin, vixetin, proanthocyanidin, apigenin, myricetin, tricetin, quercetin, naringin, kaempferol, luteolin, biflavonyl, silybin, silydianin, and silychristin. In one particularly preferred alternative, the bioflavonoid is proanthocyanidin. In another particularly preferred alternative, the bioflavonoid is silybin. Typically, the bioflavonoid is present in the composition at a concentration of from about 0.3% to about 2.0% by weight. Preferably, the bioflavonoid is present in the composition at a concentration of from 0.5% to about 1.5% by weight. More preferably, the bioflavonoid is present in the composition at a concentration of about 1.0% by weight.

In another alternative, the crosslinking agent is decorin.

Typically, the transepidermal delivery agent is selected from the group consisting of lower alkyl diols, C₁₀-C₂₀ fatty acids and esters thereof, and C₄-C₂₀ optionally substituted aliphatic alcohols. Preferably, the transepidermal delivery agent is a C₄-C₂₀ optionally substituted aliphatic alcohol. More preferably, the C₄-C₂₀ optionally substituted aliphatic alcohol is substituted with an aromatic substituent. Still more preferably, the C₄-C₂₀ optionally substituted aliphatic alcohol is benzyl alcohol or phenethyl alcohol. Most preferably, the transepidermal delivery agent is benzyl alcohol. Typically, the benzyl alcohol comprises from about 1.0% (w/w) to about 15.0% (w/w) of the composition. Preferably, the benzyl alcohol comprises from about 1.5% (w/w) to about 2.5% (w/w) of the composition. More preferably, the benzyl alcohol comprises about 2.0% (w/w) of the composition. Other transepidermal delivery agents can be used.

The composition can further comprise a chaotropic agent. Typically, the chaotropic agent is Ca(OH)₂.

The composition can further comprise a long-chain fatty acid ester of tocopherol. Preferably, the long-chain fatty acid ester of tocopherol is selected from the group consisting of tocopheryl palmitate, tocopheryl myristate, and tocopheryl stearate. More preferably, the long-chain fatty acid ester of tocopherol is tocopherol palmitate.

Typically, the topical pharmaceutically acceptable carrier comprises:

(a) water;

(b) propylene glycol;

(c) carbopol;

(d) an octyl ester of a long-chain fatty acid selected from the group consisting of octyl palmitate, octyl stearate, and octyl myristate.

(e) silicone fluid;

(f) cetearyl alcohol;

(g) triethanolamine; and

(h) at least one non-sensitizing preservative.

Typically, the octyl ester of the long-chain fatty acid is octyl palmitate. Typically, the at least one non-sensitizing preservative comprises at least one of methylparaben, ethylparaben, propylparaben, butylparaben, and diazolidinyl urea. Preferably, the at least one non-sensitizing preservative comprises methylparaben, propylparaben, and diazolidinyl urea.

The topical pharmaceutically acceptable carrier can comprise other ingredients, such as: (1) a surface-coated starch polymer; (2) a long-chain fatty acid isopropyl ester selected from the group consisting of isopropyl palmitate, isopropyl myristate, and isopropyl stearate, which is typically isopropyl palmitate; (3) a mixture of glyceryl stearate and PEG-100 stearate; (4) a long-chain fatty acid selected from the group consisting of palmitic acid, stearic acid, and myristic acid, which is typically stearic acid; (5) caprylic/capric triglyceride; (6) cetearyl alcohol; (7) caprylic/capric stearyl triglyceride; and (8) fragrance, which typically comprises natural lavender and chamomile oils.

Typically, the composition promotes the crosslinking of dermal collagen when applied to the skin of a user.

Typically, the composition promotes recovery from skin damage when applied to the skin of a user. The skin damage can be solar damage or damage from a traumatic incision, as described above.

Typically, the composition promotes regeneration of the ground substance when applied to the skin of a user. The composition promotes regeneration of GAGs, including chondroitin sulfate, dermatan sulfate, keratan sulfate, and heparan sulfate.

Typically, the composition promotes maturation of collagen when applied to the skin of a user.

Typically, the composition promotes biosynthesis of collagen when applied to the skin of a user. The biosynthesis of collagen is coordinated with its maturation to provide an effective healing process from solar damage or incisional trauma.

Typically, the composition reduces the occurrence of rhytides (wrinkles) in the skin by promoting dermal hydration.

Accordingly, therefore, another aspect of the invention is a method of repairing damage to the skin comprising applying a composition according to the present invention to skin in a quantity effective to repair damage to the skin. The damage to the skin can be solar damage or damage from incisional trauma.

Yet another aspect of the invention is a method of promoting the crosslinking of dermal collagen comprising applying a composition according to the present invention to skin in a quantity effective to promote crosslinking of dermal collagen.

Yet another aspect of the invention is a method of promoting regeneration of at least one component of the ground substance of the skin comprising applying a composition according to the present invention to skin in a quantity effective to promote regeneration of at least one component of the ground substance of the skin. The at least one component can be a glycosaminoglycan. The glycosaminoglycan can be selected from the group consisting of chondroitin sulfate, dermatan sulfate, keratan sulfate, and heparan sulfate.

Yet another aspect of the invention is a method of promoting maturation of collagen comprising applying a composition according to the present invention to skin in a quantity effective to promote maturation of collagen.

Yet another aspect of the invention is a method of promoting biosynthesis of collagen comprising applying a composition according to the present invention to skin in a quantity effective to promote biosynthesis of collagen. Typically, the biosynthesis of collagen is coordinated with its maturation to provide an effective healing process from solar damage or incisional trauma.

Yet another aspect of the invention is a method of reducing occurrence of rhytides in the skin comprising applying a composition according to the present invention to skin in a quantity effective to reduce occurrence of rhytides by promoting dermal hydration.

### BRIEF DESCRIPTION OF THE DRAWINGS

The following invention will become better understood with reference to the specification, appended claims, and accompanying drawings, where:

FIG. 1(A) is a graph showing the lack of cytotoxicity of proanthocyanidin evaluated using human skin fibroblasts grown in 10% FBS/DMEM.

FIG. 1(B) is a graph showing the cytotoxicity of glutaraldehyde evaluated similarly using human skin fibroblasts grown in 10% FBS/DMEM.

FIG. 2 is a graph showing the relationship between crosslinking effectiveness (judged by melting temperature) and proanthocyanidin concentration.

FIG. 3 are photomicrographs showing fresh tissue disintegration due to enzymatic degradation contrasted with tissue integrity of the treated tissues: (A) Untreated pericardium (control); (B) 0.5% Proanthocyanidin treated; (C) 0.625% glutaraldehyde treated; histological analysis of 24-hour digested tissue by hematoxylin/eosin staining; original magnification, ×100. is a graph showing the levels of TGF-β in skin before and after administration of the topically applied cream of the present invention.

FIG. 4 is a graph showing the results of collagenase digestion of proanthocyanidin-treated collagen sponges and controls; the solubilized collagen was quantitated by measuring hydroxyproline in solution; the data represent the percentage of the total collagen solubilized (open bar, untreated control; shaded bar, treatment with proanthocyanidin).

FIG. 5 is a graph showing the effect of proanthocyanidin on the cell proliferation and synthesis of collagen in vitro using human skin fibroblasts cultured on proanthocyanidin-treated or nontreated pericardium tissue (untreated, open bars; proanthocyanidin-treated, shaded bars); cell proliferation rate was assayed by thymidine incorporation and collagen synthesis was assayed by hydroxyproline incorporation (n=5).

FIG. 6 is a graph showing the changes in the shrinkage temperature of tissues stored in two different solutions: (a) PBS (solid line); (b) 40% ethanol/PBS (dashed line); storage temperature, 21° C.; pericardium strips were treated with 0.5% proanthocyanidin for 24 hours before being stored in the different solutions.

FIG. 7 is a series of photomicrographs showing the results of subcutaneous implantation of treated pericardium tissues; implants were retrieved after 1 and 3 weeks; (PA, proanthocyanidin; GA, glutaraldehyde; H&E staining; original magnification, ×40).

FIG. 8 shows the monomer (A) and dimer (B) forms of proanthocyanidin.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

One of the aspects of the present invention is the promotion of collagen maturation. What is meant by collagen maturation in the context of skin rejuvenation?

Collagen maturation can be defined as the process by which the fragile, soluble fibrils of collagen change into strong, insoluble fibers as they proceed from a disorganized, random, and not very useful arrangement to an organized, oriented structure providing mechanical strength to a tissue, skin in this case. The critical feature of this maturation is crosslinking.

Changes in the solubility of collagen fibers occur in newly formed collagen as it is deposited to form connective tissue structures in the body. Simultaneously, the tensile strength of fibers increases dramatically and continues to increase even after the fibers have become insoluble in neutral salt solutions. All of the physical properties of newly synthesized collagen fibers are affected by crosslinking.

The biochemistry and biology of collagen has been the subject of a great deal of study. Mammals have at least 33 genetically distinct polypeptide chains comprising at least 20 distinct collagen types that occur in different tissues of the same individual. Of these, the one that generally occurs in skin is known as Type I. Type I has the chain composition [α1 (1)]₂α2(1). A single molecule of Type I collagen is composed of three polypeptide chains with an aggregate molecular mass of about 285 kD. It has a rodlike shape with a length of about 3000 Å and a width of about 14 Å. Collagen has a distinctive amino acid composition. Nearly one-third of its residues are glycine and another 15-30% of them are proline and 4-hydroxyproline residues. Other modified residues, namely 3-hydroxyproline and 5-hydroxylysine residues, also occur in collagen but in smaller amounts. These nonstandard hydroxylated amino acids are not incorporated into collagen during polypeptide synthesis, but are produced by post-translational modification. Proline residues are converted to hydroxyproline in a reaction catalyzed by the enzyme prolyl hydroxylase. The 4-hydroxyproline residues confer stability on collagen, probably through intramolecular hydrogen bonds that involve bridging water molecules. Prolyl hydroxylase requires ascorbic acid (vitamin C) for activity.

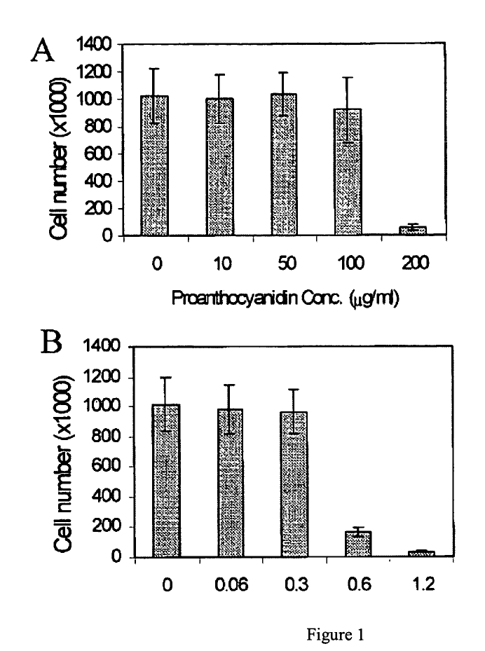

The amino acid sequence of bovine collagen α1(1), which is similar to that of other collagens, consists of monotonously repeating triplets of sequence Gly-X-Y over a continuous 1011-amino acid stretch of this 1042-residue polypeptide chain. In this repeating sequence, X is often proline and Y is often 4-hydroxyproline. The restriction of 4-hydroxyproline to the Y position in this repeating pattern stems from the specificity of prolyl hydroxylase. The modified amino acid 5-hydroxylysine is also similarly restricted to the Y position in this repeating pattern. X-ray diffraction studies have confirmed that collagen has a triple helical structure. The three polypeptide chains are parallel and wind around each other with a gentle, right-handed rope-like twist to form this triple helical structure. An individual collagen polypeptide helix has 3.3 residues per turn and a pitch of 10.0 Å. (FIG. 1) The three polypeptide chains are staggered so that the Gly, X, and Y residues in the repeating three-amino-acid sequence occur at similar levels. The staggered peptide groups are oriented so that the N—H group of each glycine residue makes a strong hydrogen bond with the carbonyl oxygen of each residue in the X position in a neighboring chain. The bulky and relatively inflexible Pro and 4-hydroxyproline residues confer rigidity on the entire assembly. This triple helical structure is responsible for its characteristic tensile strength. As with the twisted fibers of a rope, the extended and twisted polypeptide chains of collagen convert a longitudinal tensional force to a more easily supported lateral compressional force on the almost incompressible triple helix. This occurs because the oppositely twisted directions of collagen's polypeptide chains and triple helix prevent the twists from being pulled out under tension, as in ropes and cables.

Collagen is further organized into fibrils. These fibrils typically have a periodicity of 680 Å and a diameter of 100 to 200 Å. X-ray fiber diffraction studies have shown that the molecules in fibrils of Type I collagen are packed in a hexagonal array. The collagen molecules in the array are precisely staggered parallel to the fibril axis. The driving force, energetically, for the assembly of collagen molecules into a fibril is apparently provided by the added hydrophobic interactions within the fibrils. Collagen also contains covalently attached carbohydrates in amounts that range from about 0.4% to 12% by weight, depending on the collagen's tissue of origin. The carbohydrates consist mostly of glucose, galactose, and their disaccharides. They are covalently attached to collagen at its 5-hydroxylysine residues by specific enzymes. The function of the carbohydrates is not completely known, but they may be involved in directing fibril assembly.

Additional structural stability is provided in collagen by covalent crosslinking between the collagen fibrils. The crosslinking is derived from lysine and histidine side chains in reactions catalyzed by the enzyme lysyl oxidase. Lysyl oxidase is a Cu(II)-containing metalloenzyme (R. B. Rucker et al., “Copper, Lysyl Oxidase, and Extracellular Matrix Protein Cross-Linking,” Am. J. Clin. Nutr. 67 (Suppl.) 996S-1002S (1998)). In the absence of copper, the formation of lysyl and hydroxylysyl aldehydes is blocked and crosslinking of collagen, as well as of elastin, cannot occur. The first step is the oxidation of lysine residues to allysine. The next step is the aldol condensation of two allysine residues to form allysine aldol. The third step is the reaction of the allysine aldol with histidine to form an aldol-histidine product. This, in turn, can react with 5-hydroxylysine to form a Schiff base (an imine bond), which crosslinks four side chains. The crosslinked product is histidinodehydrohydroxymerodesmosine. Further information on collagen structure is provided at D. Voet & J. G. Voet, Biochemistry (3d ed., John Wiley & Sons, Hoboken, N.J., 2004), pp. 233-239, incorporated herein by this reference.

This hierarchical structure is important in understanding the process of collagen maturation. Single molecules of collagen are referred to as tropocollagen. When tropocollagen first aggregates, the force that holds the chains of tropocollagen together in their inherent arrangement is due to electrostatic bonds. When tropocollagen is first formed from procollagen, the individual a chains are held together only by hydrogen bonds. However, as compared with electrostatic bonds, hydrogen bonds are relatively weak.

Further in the process of maturation, covalent bonds are formed between the α1 and α2 chains. This is termed an intramolecular bond because it occurs within a single tropocollagen molecule. The formation of an intramolecular bond does not alter the solubility of collagen, but it does make the molecule much more stable and possibly increases its resistance to attack by enzymes. One of the most stable cross-links is the intermolecular crosslink resulting from a shift of the double bonds resulting in the Schiff base to form a ketone. This is the major force holding fibrils and their bundles together. Their presence is the chief contributing factor in the tensile strength of collagen. The formation of intramolecular and intermolecular crosslinks involving aldehyde groups occurs early in the formation of collagen fibrils. Seven distinct crosslinks have been reported in collagen, all of them dependent on oxidative deamination of lysine and hydroxylysine residues.

There are other types of bonding that further stabilize the collagen matrix. One important type of bonding that is frequently overlooked is electrostatic bonding among the protein-polysaccharide of the amorphous ground substance. This plays a role in the physical properties of the collagen fibril and may regulate the size attained by the fibril. The fibrils become covalently linked to glycoproteins. It has been suggested that fibers are formed outside of the cell in a matrix that includes a variety of mucopolysaccharides, glycoproteins, and protein-polysaccharides. Most of the sulfated mucopolysaccharides are present in the tissue in combination with protein. The high molecular weight hyaluronic acid, which exists free, facilitates the proteoglycans to imbibe water. This permits the matrix to swell and support the collagen fibers.

This dermal fibril network and cells are embedded in an amorphous extrafibrillar material that binds water and provides the hydrated consistency of the skin. While previously presumed to be biologically unstructured and largely metabolically inert, we now know that this “ground substance” is molecularly and structurally diverse, highly organized and biologically active. These biological components comprise glycosaminoglycans (GAGs) that are polysaccharides of sulfated and acetylated sugars with negative charges that bind large quantities of ions and water. Four different proteoglycan-bound GAGs are known: chondroitin sulfate, dermatan sulfate, keratan sulfate, and heparan sulfate. The structures of these proteoglycan-bound GAGs are shown at D. Voet & J. G. Voet, Biochemistry (3d ed., John Wiley & Sons, Hoboken, N.J., 2004), pp. 368-369, incorporated herein by this reference. Chondroitin sulfate occurs in two forms: chondroitin 4-sulfate and chondroitin 6-sulfate. Chondroitin 4-sulfate has N-acetyl-D-galactosamine-4-sulfate in place of the N-acetyl-D-glucosamine residues found in hyaluronic acid. Chondroitin-6-sulfate is instead sulfated at the C6 position of its N-acetyl-D-galactosamine residues. These can occur separately or in mixtures. Dermatan sulfate, which occurs frequently in skin, differs from chondroitin-4-sulfate only by an inversion of configuration about C5 of the β-D-glucuronate residues to form α-L-iduronate. This results from the enzymatic epimerization of these residues after the formation of chondroitin. The epimerization is typically incomplete, so that dermatan sulfate also contains glucuronate residues. Keratan sulfate contains alternating β(1→4)-linked D-galactose and N-acetyl-D-glucosamine-6-sulfate residues. It is typically heterogenous, as its sulfate content is variable and it contains small amounts of fucose, mannose, N-acetylglucosamine, and sialic acid. Heparan sulfate resembles heparin in its composition, and consists predominantly of alternating α(1→4) linked residues of D-iduronate-2-sulfate and N-sulfo-D-glucosamine-6-sulfate, like heparin, but has fewer N- and O-sulfate groups and more N-acetyl groups.

During disease or trauma, such as solar injury, GAG turnover is greatly enhanced. It is at this time that it becomes critical to replace these matrix components, particularly the GAGs, which seem to be most vulnerable to degradation.

The tissue specific GAGs require a source of inorganic sulfur for their synthesis. One suitable source of sulfur, utilized in the present invention, is the sulfur-containing amino acids (SAA), cysteine and methionine. These are suitable sources of sulfate used for the de novo synthesis of GAGs. These compounds are rapidly converted into free sulfate before or after absorption.

Of previously unrecognized significance in the synthesis of GAGs is that the recommended dietary allowance (RDA) for SAA (methionine and cysteine), may, in fact, underestimate the bodily needs for these mutally complementary essential nutrients, particularly during periods of increased synthesis of GAGs. Such periods of increased synthesis of GAGs are likely to occur in individuals who have suffered solar or traumatic damage to the skin, are aged, or are subject to other conditions affecting the integrity of the skin. Therefore, in addition to being building blocks for proteins such as collagen, the SM are in a class of their own as they are the primary source of sulfur used in the synthesis of many key metabolic intermediates as well as GAGs, main components of the extracellular matrix. The SM are to be included in any regimen used to restore health to solar-damaged tissue, as they facilitate dermal hydration, which aids in filling and plumping of the overlying tissue, eliminating the appearance of rhytides.

As described above, much of the evidence points to the importance of crosslinking after fibril formation as an extremely important aspect responsible for the mechanical properties of collagen, particularly tensile strength. If covalent cross-linking is important in the maturation of collagen and in rebuilding tensile strength in recovery from wounds, the introduction of crosslinks by local treatment of a healing wound with crosslinking agents might be expected to hasten the increase in tensile strength. This is not only true of incisional wounds, but also of solar damage.

It can, therefore, be inferred that the addition of copper to a topical dermatological might yield enhanced firmness of the skin because of its direct involvement in the crosslinking process. The as-yet unmet challenge, however, is making the copper biologically available to the collagen biosynthetic process in the dermis. The mere presence of topically administered copper does not result in a significant influx of copper into the dermis without the use of a transepidermal delivery agent or penetrant.

Accordingly, one aspect of the present invention is a composition for treatment of the skin comprising:

(1) methionine and cysteine in a quantity sufficient to accelerate restoration of the integrity and fullness of the skin;

(2) a mixture of essential amino acids in a quantity sufficient to accelerate restoration of the integrity and fullness of the skin, the mixture comprising isoleucine, leucine, lysine, phenylalanine, threonine, tryptophan, valine, histidine, and arginine;

(3) at least one antioxidant in a quantity sufficient to accelerate restoration of the integrity and fullness of the skin;

(4) at least one cross-linking agent in a quantity sufficient to accelerate restoration of the integrity and fullness of the skin;

(5) at least one metallic catalyst in a quantity sufficient to accelerate restoration of the integrity and fullness of the skin;

(6) at least one transepidermal delivery agent in a quantity sufficient to promote delivery of the composition to the skin; and

(7) a topical pharmaceutically acceptable carrier.

Typically, methionine is present in the composition at a concentration of from about 2% to about 35% by weight of the total amino acids. Preferably, methionine is present in the composition at a concentration of from about 2% to about 4% by weight of the total amino acids. More preferably, methionine is present at a concentration of about 3.28% by weight of the total amino acids. Typically, methionine comprises from about 0.0005% (w/w) to about 0.002% (w/w) of the composition. Preferably, methionine comprises about 0.001% (w/w) of the composition.

Typically, cysteine is present in the composition at a concentration of from about 2% to about 75% by weight of the total amino acids. Preferably, cysteine is present in the composition at a concentration of from about 25% to about 75% by weight of the total amino acids. More preferably, cysteine is present at a concentration of about 40% by weight of the amino acids. The cysteine typically comprises from about 0.01% (w/w) to about 0.4% (w/w) of the composition. Preferably, cysteine comprises about 0.2% (w/w) of the composition.

Typically, the mixture of essential amino acids (not including methionine or cysteine) comprises from about 0.005% (w/w) to about 0.5% (w/w) of the composition. Preferably, the mixture of essential amino acids (not including methionine or cysteine) comprises from about 0.1% (w/w) to about 0.4% (w/w) of the composition. More preferably, the mixture of essential amino acids (not including methionine or cysteine) comprises about 0.3% (w/w) of the composition.

The at least one antioxidant is typically selected from the group consisting of lipoic acid or a lipoic acid derivative or analogue and ascorbic acid or a derivative of ascorbic acid.

In one embodiment, the antioxidant is lipoic acid or a lipoic acid derivative or analogue. Typically, the lipoic acid or lipoic acid derivative or analogue is selected from the group consisting of lipoic acid, dihydrolipoic acid, lipoic acid esters, dihydrolipoic acid esters, lipoic acid amides, dihydrolipoic acid amides, salts of lipoic acid, and salts of dihydrolipoic acid. Lipoic acid, also known as α-lipoic acid, thioctic acid, 1,2-dithiolane-3-pentanoic acid, and 1,2-dithiolane-3-valeric acid, has the following structural formula:

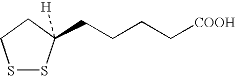

The disulfide (S—S) bond of lipoic acid is subject to reduction by chemical or biological reducing agents, leading to dihydrolipoic acid, in which the disulfide bond is replaced with two sulfhydryl (SH) groups. Because the two forms are readily interchangeable in vivo, both lipoic acid and dihydrolipoic acid, as well as their derivatives such as esters, amides, and salts, can be used in compositions according to the present invention.

If lipoic acid or an analogue or a derivative of lipoic acid is used in a composition according to the present invention, it is typically present at a concentration of from about 0.3% to about 2.0% by weight. Preferably, it is present at a concentration of from about 0.5% to about 1.5% by weight. More preferably, it is present at a concentration of 1.0% by weight.

Typically, however, the antioxidant is ascorbic acid or a derivative of ascorbic acid. Typically, the derivative of ascorbic acid is a long-chain fatty acid ester of ascorbic acid. Typically, the long-chain fatty acid ester of ascorbic acid is selected from the group consisting of ascorbyl palmitate, ascorbyl myristate, and ascorbyl stearate. Preferably, the long-chain fatty acid ester of ascorbic acid is ascorbyl palmitate. Typically, the long-chain fatty acid ester of ascorbic acid is present in the composition at a concentration of from about 0.1% (w/w) to about 0.6% (w/w). Preferably, the long-chain fatty acid ester of ascorbic acid, such as ascorbyl palmitate, is present in the composition at a concentration of about 0.3% (w/w).

Other antioxidants can be used. For example, the antioxidant can be a constituent of ginkgo. Typically, the constituent of ginkgo is selected from the group consisting of ginkgolide A, ginkgolide B, ginkgolide C, and bilobalide. In another alternative, the antioxidant is an isoflavone. Typically, the isoflavone is selected from the group consisting of genistein, genistin, 6″-0-malonylgenistin, 6″-0-acetylgenistin, daidzein, daidzin, 6″-0-malonyldaidzin, 6″-0-acetylgenistin, glycitein, glycitin, 6″-0-malonylglycitin, and 6-0-acetylglycitin. Preferably, the isoflavone is genistein or daidzein. Isoflavones can be isolated from soy or other phytochemical sources. One isolation process is disclosed in U.S. Pat. No. 6,565,912 to Gugger et al., incorporated herein by this reference.

Typically, the metallic catalyst is copper, in either its cuprous or cupric ionic forms. Preferably, the copper is in its cupric (Cu (II) ionic form), as that is the form used by the enzyme lysyl oxidase. However, the body can readily interconvert the various ionic forms of copper between the Cu(I) or Cu(II) forms. Typically, the metallic catalyst is in the form of a copper salt such as cupric acetate, cuprous acetate, cuprous chloride, cupric chloride, cuprous sulfate, cupric sulfate, or another readily soluble copper salt. As indicated above, cupric salts are preferred, such as cupric acetate, cupric chloride, or cupric sulfate. A particularly preferred cupric salt is cupric chloride. Typically, the copper salt is present in a concentration of from about 1.0% to about 5.0% by weight. Preferably, the copper salt is present in a concentration of from 1.5% to about 2.5% by weight. More preferably, the copper salt is present in a concentration of about 2.0% by weight.

The mixture of essential amino acids (other than cysteine or methionine) typically comprises:

(1) from about 5% to about 20% of isoleucine;

(2) from about 5% to about 20% of leucine;

(3) from about 10% to about 25% of lysine;

(4) from about 5% to about 20% of phenylalanine;

(5) from about 5% to about 25% of threonine;

(6) from about 5% to about 20% of tryptophan;

(7) from about 10% to about 25% of valine;

(8) from about 5% to about 20% of histidine; and

(9) from about 5% to about 20% of arginine.

Preferably, the mixture of essential amino acids (not including cysteine or methionine) comprises:

(1) about 8.48% of isoleucine;

(2) about 11.29% of leucine;

(3) about 14.68% of lysine;

(4) about 8.48% of phenylalanine;

(5) about 12.43% of threonine;

(6) about 7.91% of tryptophan;

(7) about 16.94% of valine;

(8) about 8.48% of histidine; and

(9) about 11.29% of arginine.

Typically, the crosslinking agent is a bioflavonoid. Preferably, the bioflavonoid is selected from the group consisting of quercetin, quercitrin, kaempferol, kaempferol 3-rutinoside, 3′-methoxy kaempferol 3-rutinoside, 5,8,4′-trihydroxyl-6,7-dimethoxyflavone, catechin, epicachetin, epicachetin gallate, epigallocachetin gallate, hesperidin, naringin, rutin, vixetin, proanthocyanidin, apigenin, myricetin, tricetin, quercetin, naringin, kaempferol, luteolin, biflavonyl, silybin, silydianin, and silychristin, and derivatives and glycosides of these compounds. Bioflavonoids are described, for example, in U.S. Pat. No. 6,576,271 to Nair et al., incorporated herein by this reference. Typically, the bioflavonoid is proanthocyanidin. The crosslinking activity of proanthocyanidin on collagen is described, for example, in B. Han et al., “Proanthocyanidin: A Natural Crosslinking Reagent for Stabilizing Collagen Matrices,” J. Biomed. Mater. Res. 65A: 118-124 (2003), incorporated herein by this reference. This shows that proanthocyanidin, whether in its monomeric, dimeric, or polymeric form, is an effective crosslinker of collagen and acts substantially without toxicity. The recitation of “proanthocyanidin” herein refers to any or all of the monomeric, dimeric, and polymeric forms unless otherwise specified.

Four mechanisms for interaction between proanthocyanidin and proteins have been postulated, including covalent interactions, ionic interactions, hydrogen bonding interactions, and hydrophobic interactions. The interaction between proanthocyanidin and collagen can be disrupted by detergents or hydrogen bond-weakening solvents. This suggests that proanthocyanidin and collagen complex formation involves primarily hydrogen bonding between the protein amide carbonyl in the peptide bond itself and the phenolic hydroxyl. The relatively large stability of these crosslinks compared with other polyphenols, such as tannins, suggest a structure specificity, which although encouraging hydrogen bonding also creates hydrophobic pockets. Such microenvironments, by virtue of decreasing the dielectric constant, enhance the stability of such hydrogen bonds. Hydrogen bonds that are not stabilized by adjacent hydrophobic bonds can be dissociated by treatment with aqueous buffers; however, the presence of hydrophobic bonds causes the hydrogen bonds to be relatively more stable. The relative affinity of various proteins and proanthocyanidin determined using a competitive binding assay showed that proline-rich proteins like collagen have an extremely high affinity for proanthocyanidin. Proline, an imino acid with a carbonyl oxygen adjacent to a secondary amine nitrogen, is a very good hydrogen bond acceptor; therefore, proline-rich proteins like collagen form especially strong hydrogen bonds with proanthocyanidin. Because collagen forms a helical structure, as detailed above, it enhances the accessibility of the peptide backbone for the purpose of hydrogen bonding. Hydrogen bond formation, by stabilizing the collagen fibers, is responsible for an increase in the denaturation temperature of the fixed tissue. This indicates an increase in collagen stability. Proanthocyanidin is nontoxic, and has been reported to have antibacterial, antiviral, anticarcinogenic, anti-inflammatory, and anti-allergic activities.

In another alternative, the crosslinking agent is a flavonoid that is a component of silymarin. Silymarin is an extract of the milk thistle plant, Silybium marianum. Milk thistle belongs to the aster family (Asteraceae or Compositae) that includes daisies, thistles, and artichokes. Silymarin consists of a mixture of three flavonoids that are found in the fruit, seeds, and leaves of the milk thistle plant: silybin (silybinin), silydianin, and silychristin. Silybin is the main component and is thought to have the most biological activity (Am. J. Health Syst. Pharm. 56: 1195-1197 (1999); Biochem. Pharmacol. 48: 753-759 (1994)).

When the crosslinking agent is proanthocyanidin, typically it is present in the composition at a concentration of from about 0.3% to about 2.0% by weight. Preferably, it is present in the composition at a concentration of from about 0.5% to about 1.5% by weight. More preferably, it is present in the composition at a concentration of about 1.0% by weight.

When the crosslinking agent is silybin, typically it is present in the composition at a concentration of from about 0.3% to about 2.0% by weight. Preferably, it is present in the composition at a concentration of from about 0.5% to about 1.5% by weight. More preferably, it is present in the composition at a concentration of about 1.0% by weight.

Other crosslinking agents are known and can be used. For example, the protein decorin, which interacts with collagen, can be used as a crosslinking agent. Decorin is a member of the leucine-rich repeat (LRR) protein family and is composed of a 36.5-kDa core protein substituted with one glycosaminoglycan chain on an amino-terminal Ser-Gly site (T. Krusius & E. Ruoslahti, “Primary Structure of an Extracellular Matrix Proteoglycan Core Protein Deduced from Cloned cDNA,” Proc. Natl. Acad. Sci. USA 83: 7683-7687 (1986)). The core protein contains ten leucine-rich repeats flanked by disulfide bond stabilized loops on both sides. It contains additional sites for glycosylation (N-linked glycosylation sites) within the leucine-rich repeats (Krusius & Ruoslahti (1986)). The glycosaminoglycan chain backbone is composed of repeating disaccharide units of N-acetylgalactosamine and glucuronic acid, the latter often being converted into iduronic acid through epimerization at carbon 5. As the chains are elongated, they are modified by sulfation resulting in chondroitin sulfate and dermatan sulfate respectively. The degree of epimerization and sulfation varies between tissues (F. Cheng et al., “Patterns of Uronosyl Epimerication and 4-/6-O-Sulphation in Chondroitin/Dermatan Sulfate from Decorin and Biglycan of Various Bovine Tissues,” Glycobiolociy 4: 685-696 (1994)). Decorin can also exist without glycosaminoglycan substitutions (R. Fleischmajer et al., “Decorin Interacts with Fibrillar Collagen of Embryonic and Adult Human Skin,” J. Struct. Biol. 106: 82-90 (1991)); L. O. Sampaio et al., “Dermatan Sulphate Proteoglycan from Human Articular Cartilage. Variation in Its Content with Age and Its Structural Comparison with a Small Chondroitin Sulfate Proteoglycan from Pig Laryngeal Cartilage,” Biochem. J. 254: 757-764 (1988)) or with two glycosaminoglycan substitutions (G. A. Pringle & C. M. Dodd, “Immunoelectron Microscopic Localization of the Core Protein of Decorin Near the d and e Bands of Tendon Collagen Fibrils by Use of Monoclonal Antibodies,” J. Histochem. Cytochem. 38: 1405-1411 (1988)). Decorin was shown to interact with collagen via its core protein and influence collagen fibrillogenesis (K. G. Vogel et al., “Specific Inhibition of Type I and Type II Collagen Fibrillogenesis by the Small Proteoglycan of Tendon,” Biochem. J. 223: 587-597 (1984)) and was shown to decorate the surface of collagen fibers at the d and e bands (J. E. Scott & C. R. Orford, “Dermatan Sulphate-Rich Proteoglycan Associates with Rat Tail-Tendon Collagen at the d Band in the Gap Region,” Biochem. J. 197: 213-216 (1981)). This led to the name decorin ((Krusius & Ruoslahti (1986)). Decorin interacts with fibrillar collagens and affects the fibril diameter in vitro resulting in thinner fibrils. The interactions mainly via the leucine-rich repeats 4-5 of the decorin core protein (L. Svensson et al., “Decorin-Binding Sites for Collagen Type I Are Mainly Located in Leucine-Rich Repeats 4-5,” J. Biol. Chem. 270: 20712-20716 (1995)). In addition to the fibrillar collagens I, II, III, and V (D. J. Bidanset et al., “Binding of the Proteoglycan Decorin to Collagen Type VI,” J. Biol. Chem. 267: 5250-5256 (1992); E. Hedbom & D. Heinegård, “Binding of Fibromodulin and Decorin to Separate Sites on Fibrillar Collagens,” J. Biol. Chem. 268: 27307-27312 (1993); H. C. Whinna et al., “Interaction of Heparin Cofactor II with Biglycan and Decorin,” J. Biol. Chem. 268: 3920-3924 (1993)), decorin also interacts with collagens VI, XII, and XIV (Bidanset et al. (1992); B. Font et al., “Binding of Collagen XIV with the Dermatan Sulfate Side Chain of Decorin,” J. Biol. Chem. 268: 25015-25018 (1993); B. Font et al., “Characterization of the Interactions of Type XII Collagen with Two Small Proteoglycans from Fetal Bovine Tendon, Decorin and Fibromodulin,” Matrix Biol. 15: 341-348 (1996)). Accordingly, decorin can be used as a crosslinking agent. Moreover, decorin has anti-inflammatory and anti-fibrotic properties because of its interaction with transforming growth factor-β (TGF-β), as well as its interaction with other proteins such as fibronectin, thrombospondin, the complement component C1q, and epidermal growth factor receptor EGFR. (R. A. Hildebrand et al., “Interaction of the Small Interstitial Proteoglycans Biglycan, Decorin and Fibromodulin with Transforming Growth Factor β,” Biochem. J. 302: 527-534 (1994); R. V. Iozzo et al., “Decorin Is a Biological Ligand for the Epidermal Growth Factor Receptor,” J. Biol. Chem. 274: 4489-4492 (1999); R. Krumdieck et al., “The Proteoglycan Decorin Binds C1q and Inhibits the Activity of the C1 Complex,” J. Immunol. 149: 3695-3701 (1992); R. Schmidt et al., “Interaction of Small Dermatan Sulfate Proteoglycan from Fibroblasts with Fibronectin,” J. Cell Biol. 104: 1863-1691 (1987); M. Winnemoller et al., “Interactions Between Thrombospondin and the Small Proteoglycan Decorin: Interference with Cell Attachment,” Eur. J. Cell Biol. 59: 47-55 (1992)). Still other protein crosslinking agents exist and can be used.

Typically, the transepidermal delivery agent is selected from the group consisting of lower alkyl diols, C₁₀-C₂₀ fatty acids and esters thereof, and C₄-C₂₀ optionally substituted aliphatic alcohols. Preferably, the transepidermal delivery agent is a C₄-C₂₀ optionally substituted aliphatic alcohol. More preferably, the C₄-C₂₀ optionally substituted aliphatic alcohol is substituted with an aromatic substituent. Still more preferably, the C₄-C₂₀ optionally substituted aliphatic alcohol is benzyl alcohol or phenethyl alcohol. Most preferably, the C₄-C₂₀ optionally substituted aliphatic alcohol is benzyl alcohol. However, other alcohols substituted with an aromatic substituent can alternatively be used as the transepidermal delivery agent.

Typically, when the transepidermal delivery agent is benzyl alcohol, it is present in the composition at a concentration of from about 1.0% (w/w) to about 15.0% (w/w). Preferably, the benzyl alcohol comprises from about 1.5% (w/w) to about 2.5% (w/w) of the composition. More preferably, the benzyl alcohol comprises about 2.0% (w/w) of the composition.

Other transepidermal delivery agents are known in the art and are described, for example, in U.S. Pat. No. 5,460,620 to Smith et al., incorporated herein in its entirety by this reference, and include dimethyl sulfoxide, N,N-dimethyl acetamide, 2-pyrrolidone, 1-methyl-2-pyrrolidone, Carbitol solvent (Union Carbide), propylene carbonate, 1,5-dimethyl-2-pyrrolidone, and 2-pyrrolidone-5-carboxylic acid.

Still other transepidermal delivery agents are described in U.S. Pat. No. 4,557,934 to Cooper, incorporated herein in its entirety by this reference, and include a mixture of 1-dodecylazacycloheptan-2-one in combination with a diol compound or a second N-substituted alkyl-azacycloalkyl-2-one (a “cycloketo” compound). Suitable diol compounds that form a part of this mixture include 1,2-propanediol, 1,3-propanediol, 1,2-butanediol, 1,3-butanediol, 1,4-butanediol, and 2,3-butanediol. Suitable “cycloketo” compounds are of the formula 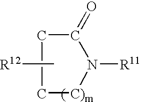
where R¹¹ is selected from the group consisting of —H, —CH₃, —C₂H₅, —C₂H₄OH, —C₃H₇, —C₃H₆OH, or —CH₂CHOHCH₂OH, R¹² is —H, —CH₃, —C₂H₅, —C₃H₇, or —C₄H₉, and m is an integer from 0 to 2.

Still other transepidermal delivery agents are described in U.S. Pat. No. 4,946,870 to Partain, III, et al., incorporated herein in its entirety by this reference, and include aminopolysaccharides such as chitosonium polymers and covalent derivatives of chitosan prepared by the reaction of chitosan with one or more electrophilic reagents such as ethylene oxide, propylene oxide, glycidol, C₁-C₂₄ alkyl halides, glycidyl C₁-C₂₄ trialkylammonium salts, 3-chloro-2-hydroxypropyl ammonium salts, 1,3-propanesultone, haloacetates, succinic anhydride, maleic anhydride, carboxylic acyl halides, N-carboxy-α-amino acid anhydrides, and other electrophilic reagents.

Yet other transepidermal delivery agents are described in U.S. Pat. No. 6,765,001 to Gans et al., incorporated herein in its entirety by this reference, and include diisopropyl adipate, dimethyl isosorbide, propylene glycol, and 1,2,6-hexanetriol.

Still other transepidermal delivery agents are described in U.S. Pat. No. 6,586,473 to Gans et al., incorporated herein in its entirety by this reference, and include dioctyl maleate, propylene carbonate, and diisopropyl sebacate.

Still other transepidermal delivery agents are known in the art.

Suitable topical pharmaceutically acceptable carriers are disclosed in U.S. Pat. No. 5,935,994, incorporated herein by this reference. The carrier is typically in the form of a cream base that is compatible with all of the ingredients as far as stability is concerned.

Typically, the topical pharmaceutically acceptable carrier comprises:

(1) water;

(2) propylene glycol;

(3) carbopol;

(4) an octyl ester of a long-chain fatty acid selected from the group consisting of octyl palmitate, octyl stearate, and octyl myristate.

(5) silicone fluid;

(6) cetearyl alcohol;

(7) triethanolamine; and

(8) at least one non-sensitizing preservative.

A suitable silicone fluid is a silicone fluid with a viscosity of 200 cs.

A suitable preparation of carbopol is Carbopol 940. Other carboxypolymethylene polymers are known in the art, such as Carbomer polymers, and can be used.

Triethanolamine is a buffer and can be replaced by other buffers that can buffer the topical pharmaceutically acceptable carrier to a physiological pH.

Typically, the octyl ester of a long-chain fatty acid is selected from the group consisting of octyl palmitate, octyl stearate, and octyl myristate is octyl palmitate.

The topical pharmaceutically acceptable carrier can further comprise other, optional, ingredients.

For example, the topical pharmaceutically acceptable carrier can further comprise a surface-coated starch polymer. A suitable surface-coated starch polymer is Dryflo PC, marketed by National Starch.

The topical pharmaceutically acceptable carrier can also further comprise a long-chain fatty acid isopropyl ester selected from the group consisting of isopropyl palmitate, isopropyl myristate, and isopropyl stearate. Typically, the long-chain fatty acid isopropyl ester is isopropyl palmitate.

The topical pharmaceutically acceptable carrier can also further comprise a mixture of glyceryl stearate and PEG-100 stearate. A suitable mixture of glyceryl stearate and PEG-100 stearate is Arlacel 165.

The topical pharmaceutically acceptable carrier can also further comprise a long-chain fatty acid selected from the group consisting of palmitic acid, stearic acid, and myristic acid. Typically, the long-chain fatty acid is stearic acid.

The topical pharmaceutically acceptable carrier can also further comprise caprylic/capric triglyceride. A suitable caprylic/capric triglyceride is Miglyol 812.

The topical pharmaceutically acceptable carrier can also further comprise cetearyl alcohol.

The topical pharmaceutically acceptable carrier can also further comprise caprylic/capric stearyl triglyceride. A suitable caprylic/capric stearyl triglyceride is Softisan 378.

The topical pharmaceutically acceptable carrier can also further comprise fragrance. Typically, the fragrance comprises natural lavender and chamomile oils. However, other fragrances are well known in the art of preparing products suitable for application to the skin, and can be used as alternatives.

Typically, the non-sensitizing preservative of the topical pharmaceutically acceptable carrier comprises at least one of methylparaben, ethylparaben, propylparaben, butylparaben, and diazolidinyl urea. Preferably, the non-sensitizing preservative comprises methylparaben, propylparaben, and diazolidinyl urea. A suitable preparation of diazolidinyl urea is Germall 2.

Other ingredients are well known in the art of preparing cosmetics and other products suitable for application for the skin, and can be used in the topical pharmaceutically acceptable carrier. For example, other lipid-soluble components can be used in addition to or in place of the caprylic/capric triglycerides. Such components can include but are not limited to: steareth-2; steareth-21; polyglyceryl-3 beeswax; a branched-chain carboxylic acid ester of a branched-chain alcohol selected from the group consisting of isononyl isononanoate, isodecyl isononanoate, isooctyl isononanoate, isononyl isooctanoate, isodecyl isooctanoate, isooctyl isooctanoate, isononyl isodecanoate, isooctyl isodecanoate, and isodecyl isodecanoate; acrylates/C₁₀-C₃₀ alkyl acrylates cross-polymers; methylgluceth-20; a glyceryl ester of a long-chain fatty acid selected from the group consisting of glyceryl monostearate, glyceryl monopalmitate, and glyceryl monoarachidate; hydrogenated vegetable oil; squalane; C₁₂-C₁₅ alkylbenzoates; di-C₁₂-C₁₅ alkylfumarate; cholesterol; lanolin alcohol; octyldodecanol; isostearic acid; a branched-chain neopentanoate selected from the group consisting of octyldodecyl neopentanoate, heptyldodecyl neopentanoate, nonyidodecyl neopentanoate, octylundecyl neopentanoate, heptylundecyl neopentanoate, nonylundecyl neopentanoate, octyltridecyl neopentanoate, heptyltridecyl neopentanoate, and nonyltridecyl neopentanoate; an arachidyl ester of a short-chain carboxylic acid selected from the group consisting of arachidyl propionate, arachidyl acetate, arachidyl butyrate, and arachidyl isobutyrate; jojoba oil; a myristyl ester of a long-chain fatty acid selected from the group consisting of myristyl myristate, myristyl laurate, and myristyl palmitate; bisabolol; hydrogenated jojoba oil; jojoba esters; methylgluceth-20 sesquistearate; PPG-14 butyl ether; PPG-15 stearyl ether; PPG-1-isoceteth-3-acetate; laureth-2-benzoate; diisostearyl dimer dilinoleate; a long-chain cis-monounsaturated fatty acid ester of a medium-chain alcohol; a medium-chain saturated carboxylic acid ester of a long-chain alcohol; hydrogenated soy glycerides; a long-chain fatty acid ester of cetyl alcohol selected from the group consisting of cetyl palmitate, cetyl stearate, and cetyl myristate; palm kernel oil; and palm oil.

In addition, the topical pharmaceutically acceptable carrier can further comprise other ingredients that are generally used in the cosmetic art and in the art of skin preparations. These ingredients include, but are not limited to:

(1) plant extracts, such as horsetail extract, horse chestnut extract, rose extract, or lavender extract;

(2) a long-chain fatty acid ester of retinol or a retinol derivative or analogue wherein the acyl moiety of the ester is selected from the group consisting of myristic acid, palmitic acid, and stearic acid; and

(3) a sunscreen, which can be at least one compound selected from the group consisting of octyl methoxycinnamate, p-aminobenzoic acid, ethyl p-aminobenzoate, isobutyl p-aminobenzoate, glyceryl p-aminobenzoate, p-dimethylaminobenzoic acid, methyl anthranilate, menthyl anthranilate, phenyl anthranilate, benzyl anthranilate, phenylethyl anthranilate, linalyl anthranilate, terpinyl anthranilate, cyclohexenyl anthranilate, amyl salicylate, phenyl salicylate, benzyl salicylate, menthyl salicylate, glyceryl salicylate, dipropyleneglycol salicylate, methyl cinnamate, benzyl cinnamate, .alpha.-phenyl cinnamonitrile, butyl cinnamoylpyruvate, umbelliferone, methylacetoumbelliferone, esculetin, methylesculetin, daphnetin, esculin, daphnin, diphenylbutadiene, stilbene, dibenzalacetone, benzalacetophenone, sodium 2-naphthol-3,6-disulfonate, sodium 2-naphthol-6,8-disulfonate, dihydroxynaphthoic acid, salts of dihydroxynaphthoic acid, o-hydroxybiphenyldisulfonates, p-hydroxybiphenyldisulfonates, 7-hydroxycoumarin, 7-methylcoumarin, 3-phenylcoumarin, 2-acetyl-3-bromoindazole, phenylbenzoxazole, methylnaphthoxazole, arylbenzothiazoles, quinine bisulfate, quinine sulfate, quinine chloride, quinine oleate, quinine tannate, 8-hydroxyquinoline salts, 2-phenylquinoline, hydroxy-substituted benzophenones, methoxy-substituted benzophenones, uric acid, vilouric acid, tannic acid, tannic acid hexaethylether, hydroquinone, oxybenzone, sulisobenzone, dioxybenzone, benzoresorcinol, 2,2′,4,4′-tetrahydroxybenzo-phenone, 2,2′-dihydroxy-4,4′dimethoxybenzophenone, octabenzone, 4-isopropyldibenzoylmethane, butylmethoxydibenzoylmethane, etocrylene, and 4-isopropyldibenzoylmethane.

Other ingredients can optionally be included in the topical pharmaceutically acceptable carrier.

The composition can optionally further include an effective concentration of a chaotropic agent to increase the efficiency of crosslinking. A suitable chaotropic agent is calcium hydroxide (Ca(OH)₂). Other chaotropic agents are well known in the art.

Compositions according to the present invention can be prepared by standard procedures, such as those typically used for cosmetic preparations and pharmaceutical compositions intended for topical application to the skin. These procedures include mixing techniques, including both manual and mechanical mixing, and including homogenization mixing and sweep mixing. The mixing techniques to be used can be chosen by one of ordinary skill in the art based on variables such as the viscosity of the components to be mixed and the volume of those components, as well as the relative proportion of lipid-soluble and water-soluble ingredients. Typically, the individual active ingredients are added sequentially, and benzyl alcohol or the other transepidermal delivery agent is added to the desired final concentration. Water and oil phases are heated separately to 70° C., blended, and cooled with normal mixing.

One preferred formulation of the topical pharmaceutically acceptable carrier is shown in Table 1.

TABLE 1
FORMULATION OF TOPICAL PHARMACEUTICALLY
ACCEPTABLE CARRIER

| Ingredient | % (w/w) |
|---|---|
| Propylene glycol | 2.23 |
| Carbopol | 1.12 |
| Surface coated starch polymer | 0.56 |
| Octyl palmitate | 1.12 |
| Isopropyl palmitate | 2.23 |
| Silicone fluid | 2.23 |
| Glyeryl stearate/PEG-100 stearate | 2.23 |
| Cetearyl alcohol | 1.12 |
| Stearic acid | 0.56 |
| Triethanolamine | 0.28 |
| Caprylic/capric triglyceride | 2.23 |
| Caprylic/capric stearyl triglyceride | 0.56 |
| Natural lavender/chamomile oils | 0.22 |
| Methylparaben | 0.22 |
| Propylparaben | 0.06 |
| Diazolidinyl urea | 0.22 |
| Water | q.s. to 100 |

Typically, the composition promotes the crosslinking of dermal collagen when applied to the skin of a user.

Typically, the composition promotes recovery from skin damage when applied to the skin of a user. The skin damage can be solar damage or damage from a traumatic incision, as described above.

Typically, the composition promotes regeneration of the ground substance when applied to the skin of a user. The composition promotes regeneration of GAGs, including chondroitin sulfate, dermatan sulfate, keratan sulfate, and heparan sulfate.

Typically, the composition promotes maturation of collagen when applied to the skin of a user.

Typically, the composition promotes biosynthesis of collagen when applied to the skin of a user. The biosynthesis of collagen is coordinated with its maturation to provide an effective healing process from solar damage or incisional trauma.

Typically, the composition reduces the appearance of rhytides (wrinkles) in the skin by promoting dermal hydration.

Compositions according to the present invention can be applied by users as they would apply standard cosmetics or other creams, once or more daily, depending on age, skin condition, and other variables readily apparent to the user.

Accordingly, therefore, another aspect of the invention is a method of repairing damage to the skin comprising applying a composition according to the present invention to skin in a quantity effective to repair damage to the skin. The damage to the skin can be solar damage or damage from incisional trauma.

Yet another aspect of the invention is a method of promoting the crosslinking of dermal collagen comprising applying a composition according to the present invention to skin in a quantity effective to promote crosslinking of dermal collagen.

Yet another aspect of the invention is a method of promoting regeneration of at least one component of the ground substance of the skin comprising applying a composition according to the present invention to skin in a quantity effective to promote regeneration of at least one component of the ground substance of the skin. The at least one component can be a glycosaminoglycan. The glycosaminoglycan can be selected from the group consisting of chondroitin sulfate, dermatan sulfate, keratan sulfate, and heparan sulfate.

Yet another aspect of the invention is a method of promoting maturation of collagen comprising applying a composition according to the present invention to skin in a quantity effective to promote maturation of collagen.

Yet another aspect of the invention is a method of promoting biosynthesis of collagen comprising applying a composition according to the present invention to skin in a quantity effective to promote biosynthesis of collagen. Typically, the biosynthesis of collagen is coordinated with its maturation to provide an effective healing process from solar damage or incisional trauma.

Yet another aspect of the invention is a method of reducing appearance of rhytides in the skin comprising applying a composition according to the present invention to skin in a quantity effective to reduce appearance of rhytides by promoting dermal hydration. Compositions according to the present invention can also be used prophylactically to reduce the occurrence of rhytides.

The invention is illustrated by the following Example. This Example is included for illustrative purposes only, and is not intended to limit the invention.

### EXAMPLE

### Effect of Proanthocyanidin on Stability of Collagen

Materials and Methods

Cytotoxicity

NIH 3T3 cells were used in these studies. Cells were cultured in 24-well plates at a density of 5×10⁶ cells/well in 10% FBS/DMEM overnight. The medium was then replaced with complete medium supplemented with proanthocyanidin (MegaNatural), provided by Polyphenolics (Madera, Calif.), in a concentration of 0, 20, 100, or 200 μg/mL, or glutaraldehyde (GA) in a concentration of 0, 0.1, 0.5, 1.0, or 5.0 μg/mL. Cells were incubated for 72 hours before cell counting and morphological studies.

Fixation Process

Fresh bovine tendon, pericardium strips, and processed collagen sponges (prepared with bovine tendon atelopeptide-collagen) were fixed with either 0.5% proanthocyanidin PBS solution (pH 7.4) or 0.625% GA/PBS solution for 48 h at room temperature.

In Vitro Enzymatic Degradation

Proanthocyanidin-fixed tendon tissue together with fresh controls were digested with 0.2% collagenase (Worthington Biochemicals, NJ), at pH 7.4 for 24 hr at 37° C. Tissue integrity was checked at the end of the incubation using a standard histological method (hematoxylin-eosin (H&E)). To quantitate enzyme digestion rate, 500 mg of both Type I collagen sponges treated with proanthocyanidin or untreated was digested with 15 mL of 0.2% collagenase in PBS solution at 37° C. At predetermined intervals, 1.0 mL solution was taken out and filtered through an 0.45-μM cellulose filter to separate solubilized collagen from insolubilized matrix. The amount of solubilized collagen was determined after total acid hydrolysis in 6N HCl for 24 h at 100° C. by measuring hydroxyproline (R. Kultan et al., “Collagen Treated with (+)-Catechin Becomes Resistant to the Action of Mammalian Collagenase,” Experientia 37: 221-223 (1981)). The results are expressed as a percentage of the total collagen solubilized.

Melting Temperature Measurement

Melting temperature (Tm) has been extensively used as an indicator of the amount of crosslinking in biopolymers. The fixed tissues and fresh tissues were assayed for their melting temperature by heating tissue strips (1×2 cm², n=3). The melting temperature was recorded when tissues started to shrink.

The Stability of Proanthocyanidin-Treated Tissue

After 48-h fixation in proanthocyanidin, tissue was incubated in PBS containing 0.5% sodium azide solution at 37° C. for preservation. For prolonged storage, tissues were kept in 40% ethanol/PBS (controls). After different time intervals, the shrinkage temperature of the tissues (Tm) was measured after thorough rinsing.

In Vitro Cell Culture

Discs, 15 mm in diameter, were punched out from PA-treated bovine pericardium and inserted into the bottom of 24-well plates, After washing and equilibrating with PBS, human skin fibroblasts, 8×10⁴/well, third passage (kindly provided by Dr. Warren Garner, University of Southern California) were placed on top of the tissues. After 48 h of culture in 10% FBS/DMEM, the medium was changed to labeling medium ([³H]thymidine, 10 μCi/mL, 0.5% FBS/DMEM) followed by a 24-h labeling period. Cells proliferation was assayed and collagen synthesis recorded as [³H]OH-proline incorporation in a culture medium containing 25 μg/mL ascorbic acid, 25 μg/mL β-APN and 25 μCi/mL [³H]proline in 0.5% FBS/DMEM after labeling for 48 h.

Subcutaneous Implantation

Three-week-old Fischer 344 rats were obtained from Harlan Sprague Dawley, Inc. (Indianapolis, Ind.). NIH and University of Southern California IACUC guidelines for the care and use of laboratory animals were observed. Proanthocyanidin or GA-treated collagen sponges (1×1 cm²) and bovine pericardium (1×2 cm²) were implanted subcutaneously on the back of the animal (n=4). Similar materials without treatment were implanted as controls. The samples were retrieved after 3 and 6 weeks postoperation, and samples were processed for H&E and von Kossa staining, the latter to determine the extent of calcification.

Results

Cytotoxicity

After a 72-h incubation, cells grown in medium supplemented with 0-100 μg/mL proanthocyanidin proliferate normally (FIG. 1(A)). No cytotoxicity of proanthocyanidin can be observed until the concentration approaches 200 μg/mL. On the other hand, GA exhibited obvious cytotoxicity, even at the concentration of 0.6 μg/mL (FIG. 1(B)). The potential cytotoxicity may arise from residues of unreacted or degraded crosslinking agents. In the study reported in this Example, fibroblasts could grow with a high concentration of proanthocyanidin in the medium (200 μg/mL), whereas cells could not survive when the GA concentration was >0.6 μg/mL. These results indicated that any polyphenolic residues, either from unreacted proanthocyanidin or from degradation of crosslinked materials, had little toxic effects.

Physicochemical Properties of Proanthocyanidin-Treated Tissue

When treated with 0.5% proanthocyanidin, tissues turned brownish in color because of the color of the solution. Table 2 presents the melting temperature of different tissues treated with proanthocyanidin (PA) or GA. For both tendon and pericardium, melting temperatures increased dramatically in the proanthocyanidin group compared with fresh controls (p<0.05).

TABLE 2
Differences in Melting Temperatures of Fresh
and Treated Specimens Obtained from Two different
Sources (Tendon) and Pericardium)

| Tissue | Control | PA^{a} | GA^{b} |
|---|---|---|---|
| Tendon | 55 ± 0.5 | 80 ± 1.5 | 84 ± 1.0 |
| Pericardium | 70 ± 1.0 | 91 ± 2.0 | 94 ± 1.5 |

Specimens were treated in corresponding solutions for 48 hours (n = 3).
^{a}Proanthocyanidins, 0.5% in PBS.
^{b}Glutaraldehyde, 0.625% in PBS.

The increase in Tm from 66 to 86° C. of bovine pericardium upon reaction with proanthocyanidin showed that effective crosslinking of collagen occurred under mild conditions. The crosslinking is likely to arise from hydrogen bonds formed between the polyphenolic structure of proanthocyanidin and collagen chains that are in their physiological triple helical conformation. Therefore, these findings are readily applicable to a broad range of collagens.

Concentration of Proanthocyanidin and Crosslinking Efficiency

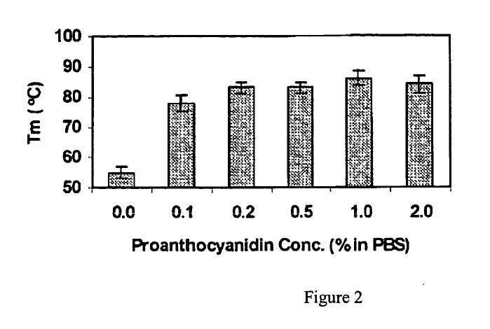

By comparing concentration of proanthocyanidin with melting temperatures (FIG. 2), it was found that 0.5% was optimal for maximally crosslinking the tissue. However, the concentration of the crosslinking solution used is an important consideration not only for the degree of crosslinking, but also for crosslinking efficiency. When 1.0% proanthocyanidin was used to great bovine tendon, the center of this rather large tendon was not fixed well and was readily digested by collagenase. It was found that lower concentrations of fixative penetrated into the tissues more readily, thus increasing the efficiency of fixation particularly when 0.05M Ca(OH)₂ was added to the fixation solution. Ca(OH)₂, a chaotropic agent, at this concentration, appears to help proanthocyanidin penetrate while keeping the tissue from swelling to any significant degree.

In Vitro Enzymatic Degradation

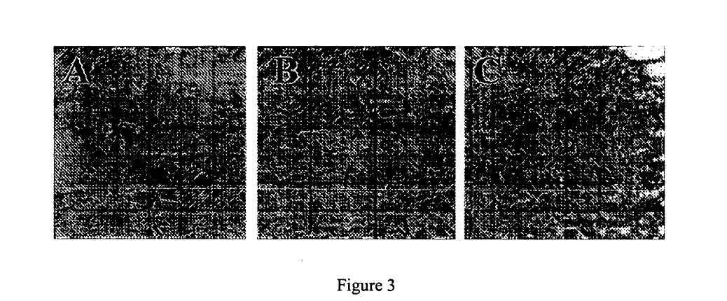

FIG. 3 presents the histological appearance of fresh, proanthocyanidin-fixed, and GA-fixed bovine pericardium, stained with H&E, after 24 h of collagenase digestion ((A); untreated pericardium (control); (B) 0.5% proanthocyanidin treated; (C) 0.625% GA treated; histological analysis of 24-h digested tissue by H&E staining; original magnification, ×100). In all instances, fresh tissues disintegrated into small pieces. In contrast, the collagen fibril structure of the GA- and proanthocyanidin-treated tissues remained intact.

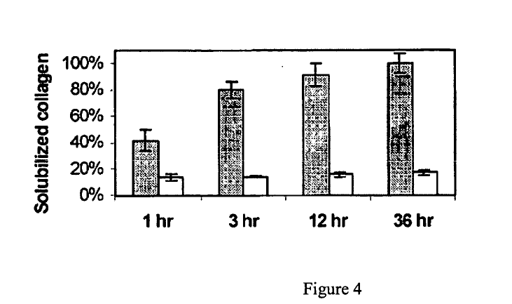

FIG. 4 illustrated the enzyme digestion rate by checking the amount of solubilized collagen at different digestion times when proanthocyanidin-treated collagen sponges and controls were digested(1 h, 3 h, 12 h, 36 h; open bar, untreated control; shaded bar, treated with proanthocyanidin). The solubilized collagen was quantitated by measuring hydroxyproline in solution. Fresh pericardium was completely digested after 36 h, whereas proanthocyanidin-treated tissues remained intact after collagenase treatment.

Cell Proliferation and Collagen Synthesis on the Surface of Treated Pericardium Matrices

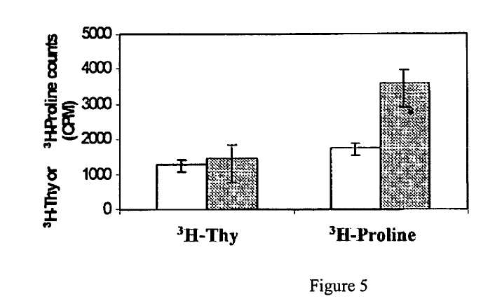

There are no significant differences in cell proliferation rates of human skin fibroblasts cultured on proanthocyanidin-treated or nontreated fresh bovine pericardium. On the other hand, proanthocyanidin treatment seems to enhance the cell's ability to deposit collagen (p<0.005; FIG. 5). In FIG. 5, cell proliferation rates and collagen synthesis of human fibroblasts cultured on proanthocyanidin-treated pericardium tissue (untreated, open bars; proanthocyanidin-treated, shaded bars). Cell proliferation rate was assayed by thymidine incorporation and collagen synthesis was assayed by hydroxyproline incorporation (n=5).

Stability of Proanthocyanidin-Treated Tissue

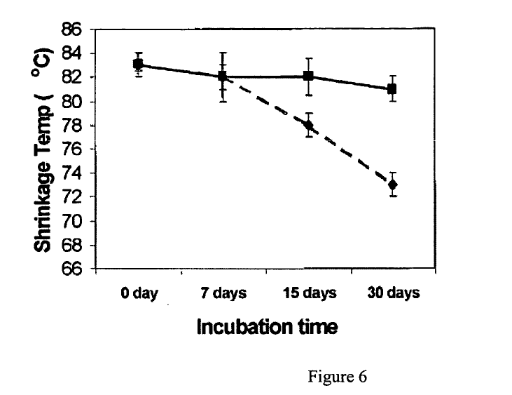

The stability of proanthocyanidin-induced crosslinking was evaluated under physiological conditions in vitro. When tissue was stored in PBS at 37° C. for 30 days, the hydrogen bonds were destabilized and shrinkage temperature began to decrease, but when the dielectric constant of the solution was lowered by adding 40% ethanol to the PBS, crosslinks remained stable and the shrinkage temperature remained constant (FIG. 6), reflecting the participation of hydrogen bonding in this process. In FIG. 6, the shrinkage temperature in PBS is shown by the solid line; the shrinkage temperature in 40% ethanol/PBS is shown by the dashed line. Pericardium strips were treated with 0.5% proanthocyanidin for 24 h before being stored in the different solutions.

Subcutaneous Implantation

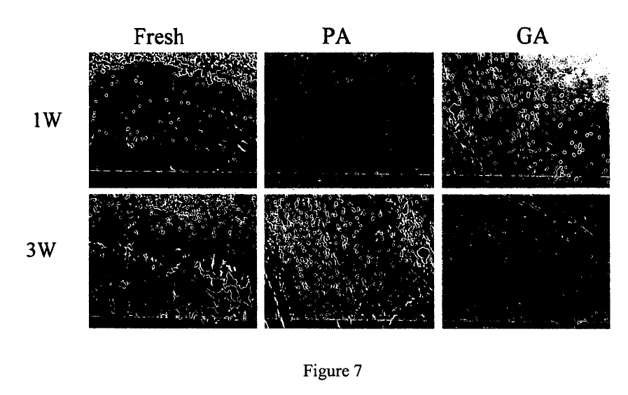

One week postoperatively, untreated pericardium (controls), gives rise to a notable inflammatory reaction, whereas proanthocyanidin-treated specimens show cell invasion and ingrowth. Glutaraldehyde-treated samples, after being thoroughly rinsed, exhibit a lesser inflammatory reaction. Three weeks postoperatively, control tissues start to disaggregate, whereas the proanthocyanidin- and GA-fixed tissues retain their integrity. The proanthocyanidin treated specimen appears to be the most tissue compatible. New fibroblasts penetrate and proliferate inside the tissue (FIG. 7).

Six weeks postoperatively, control tissue could not be retrieved because it had been completely degraded. On the other hand, the proanthocyanidin-treated specimens were just starting to degrade, whereas GA-treated tissues were still intact. Von Kossa staining, which specifically indicates the presence of calcification, showed that there was no calcification in proanthocyanidin-treated tissues, whereas GA-treated tissues exhibited dystrophic calcification (data not shown) (D. T. Cheung et al., “Mechanism of Crosslinking of Proteins by Glutaraldehyde. III. Reaction with Collagen in Tissues,” Connect. Tissue Res. 13: 109-115 (1985); M. E. Nimni et al., “Biochemical Differences Between Dystrophic Calcification of Cross-Linked Collagen Implants and Mineralization During Bone Induction,” Calcif. Tissue Int. 42: 313-320 (1988)). In FIG. 7, tissues are shown at 1 and 3 weeks postoperatively; implants were retrieved at those time points (PA, proanthocyanidin; GA, glutaraldehyde; H&E staining; original magnification, ×40).

Discussion

Collagen has been used extensively in the manufacturing of bioprostheses and in the design of tissue engineered scaffolds. Of course, as indicated above, there are many other circumstances in which the stability and maturation of collagen is of critical importance. Fixation of biological tissues can reduce their antigenicity and increase their resistance to enzymatic degradation after implantation (M. E. Nimni et al., “Chemically Modified Collagen: A Natural Biomaterial for Tissue Replacement,” J. Biomed. Mater. Res. 21: 741-771 (1987); L. L. Huang-Lee & M. E. Nimni, “Crosslinked CNBr-Activated Hyaluronan-Collagen Matrices: Effects on Fibroblast Contraction,” Matrix Biol. 14: 147-157 (1994)). Various crosslinking reagents, which include formaldehyde, glutaraldehyde (D. T. Cheung et al., “Mechanism of Crosslinking of Proteins by Glutaraldehyde. IV: In Vitro and in Vivo Stability of a Crosslinked Collagen Matrix,” Connect. Tissue Res. 25: 27-34 (1990)), epoxy compounds, and carbodiimide, have been used, but all have drawbacks, including toxicity, crosslinking rates that are difficult to control, and instability. Therefore, it is desirable to find an agent that can crosslink collagen and that can assist in the maturation of collagen while retaining its stability.

Proanthocyanidin (PA) compounds appear to be good candidates to fulfill such a role. They are naturally occurring plant metabolites widely available in fruits, vegetables, nuts, seeds, flowers, and barks (S. S. Joshi et al., “The Cellular and Molecular Basis of Health Benefits of Grape Seed Proanthocyanidin Extract,” Curr. Pharm. Biotechnol. 2: 187-200 (2001); A. M. Fine, “Oligomeric Proanthocyanidin Complexes: History, Structure, and Phytopharmaceutical Applications,” Altern. Med. Rev. 5: 144-151 (2000)). Proanthocyanidins are part of a specific group of polyphenolic compounds and belong to the category known as condensed tannins.

Proanthocyanidins were found to increase collagen synthesis and accelerate the conversion of soluble collagen to insoluble collagen during development (C. N. Rao et al., “Bioflavonoid-Mediated Stabilization of Collagen in Adjuvant-Induced Arthritis,” Scand. J. Rheumatol. 12: 39-42 (1983); G. Cetta et al., “Influence of Flavonoid-Copper Complexes on Cross Linking in Elastin,” Ital. J. Biochem. 26: 317-327 (1977)). In skin fibroblast cultures derived from Marfan patients and those of patients with Ehler-Danlos Type V, the excessive solubility fo collagen can be corrected by the addition of a synthetic proanthocyanidin to the culture medium (G. Francis et al., “Abnormally Soluble Collagen Produced in Fibroblasts Cultures,” Experientia 32: 691-693 (1976)) They also inhibit the catabolism of soluble collagen in animal studies (R. Kuttan et al., “Collagen Treated with (+)-Catechin Becomes Resistant to the Action of Mammalian Collagenase,” Experientia 37: 221-223 (1981)), stimulate normal skin fibroblast production, and increase the synthesis of extracellular matrix, including collagen and fibronectin (S. J. Kim et al., “Effects of Flavonoids of Ginkgo biloba on Proliferation of Human Skin Fibroblast,” Skin Pharmacol. 10: 200-205 (1997)). Proanthocyanidins are natural products with polyphenolic structures that have the potential to give rise to stable hydrogen bonded structures and generate nonbiodegradable collagen matrices. Furthermore, proanthocyanidins are widely used as natural antioxidants and free-radical scavengers, and have proven to be safe in different clinical applications and as dietary supplements (R. M. Facino et al., “Diet Enriched with Procyanidins Enhances Antioxidant Activity and Reduces Myocardial Post-Ischaemic Damage in Rats,” Life Sci. 64: 627-642 (1999)). The lack of acute and subacute toxicity of proanthocyanidin has been confirmed in rats (K. C. Sabino et al., “In Vitro and In Vivo Toxicological Study of the Pterodon pubescens Seed Oil,” Toxicol. Lett. 108: 27-35 (1999); X. Ye et al., “The Cytotoxic Effects of a Novel IH636 Grape Seed Proanthocyanidin Extract on Cultured Human Cancer Cells,” Mol. Cell. Biochem. 196: 99-108 (1999)). The free-radical-scavenging ability of proanthocyanidins have been well documented and have commanded the most attention (D. Bagchi et al., “Free Radicals and Grape Seed Proanthocyanidin Extract: Importance in Human Health and Disease Prevention,” Toxicology 148: 187-197 (2000)).

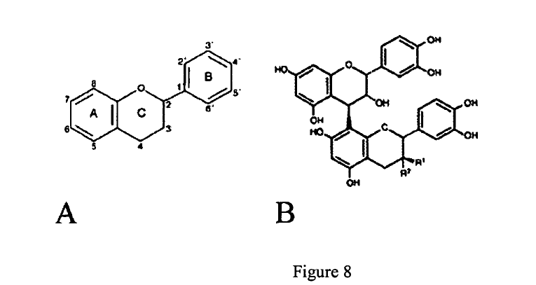

In proanthocyanidin, a benzene-pyran-phenolic acid molecular nucleus is the core structure of the oligomeric form (FIG. 8) and the polymer forms of such a complex (K. Billiar et al., “Effects of Carbodiimide Crosslinking Conditions on the Physical Properties of Laminated Intestinal Submucosa,” J. Biomed. Mater. Res. 56: 101-106 (2001)) FIG. 8(B) shows the dimer form.

Four mechanisms for interaction between proanthocyanidin and proteins have been postulated, including covalent interactions (W. S. Pierpoint, “o-Quinones Formed in Plant Extracts. Their Reactions with Amino Acids and Peptides,” Biochem. J. 112: 609-616 (1969)), ionic interactions (W. D. Loomis, “Overcoming Problems of Phenolics and Quinones in the Isolation of Plant Enzymes and Organelles,” Methods Enzymol. 31: 528-544 (1974)), hydrogen bonding interactions, and hydrophobic interactions. The interactions between proanthocyanidin and collagen can be disrupted by detergents or hydrogen-bond-weakening solvents, suggesting that proanthocyanidin and collagen complex formation involves primarily hydrogen bonding between the protein amide carbonyl and the phenolic hydroxyl (A. E. Hagerman & K. M. Klucher, “Tannin-Protein Interactions,” Prog. Clin. Biol. Res. 213: 67-76 (1986)). The relatively large stability of these crosslinks compared with those between proteins and other phenols such as tannins suggests a structure specificity, which, although encouraging hydrogen bonding, also creates hydrophobic pockets. Such microenvironments, by virtue of decreasing the effective dielectric constant, enhance the stability of hydrogen bonds. Hydrogen bonds that are not stabilized by adjacent hydrophobic bonds can be dissociated by treatment with aqueous buffers. Alcohols, on the other hand, by decreasing the dielectric constant of the medium, also stimulate proanthocyanidin-collagen interactions. Therefore, in the experiments reported in this Example, the crosslinked matrices were therefore maintained in a 40% alcohol solution for long-term storage.

The relative affinity of various proteins and proanthocyanidin determined using a competitive binding assay (A. E. Hagerman & L. G. Butler, “The Specificity of Proanthocyanidin-Protein Interactions,” J. Biol. Chem. 256: 4494-4497 (1981)) showed that proline-rich proteins like collagen have an extremely high affinity for proanthocyanidin. Proline, an imino acid with a carbonyl oxygen adjacent to a secondary amine nitrogen, is a very good hydrogen bond acceptor; therefore, proline-rich proteins like collagen form especially strong hydrogen bonds with proanthocyanidin. Because collagen is a helical structure, as outlined above, it enhances the accessibility of the peptide backbone for the purpose of hydrogen bonding. Hydrogen bond formation, by stabilizing the collagen fibers, is responsible for the increase in the denaturation temperature of the fixed tissue. The shrinkage temperature (denaturation temperature) of the fixed bovine pericardium increased from 66 to 86° C., thereby demonstrating the efficacy of the crosslinking by proanthocyanidin.

Chronic cytotoxicity is always of primary concern when agents that penetrate the skin are being evaluated for their effectiveness. Proanthocyanidins are widely used as food supplement, and their lack of toxicity has been extensively demonstrated. In addition, proanthocyanidins have been reported to possess antibacterial, antiviral, anticarcinogenic, anti-inflammatory, and anti-allergic activities (X. Ye et al., “The Cytotoxic Effects of a Novel IH636 Grape Seed Proanthocyanidin Extract on Cultured Human Cancer Cells,” Mol. Cell. Biochem. 196: 99-108 (1999); D. Bagchi et al., “Free Radicals and Grape Seed Proanthocyanidin Extract: Importance in Human Health and Disease Prevention,” Toxicology 148: 187-197 (2000); A. Scalbert et al., “Proanthocyanidins and Human Health: Systemic Effects and Local Effects in the Gut,” Biofactors 13: 115-120 (2000)). A cytotoxicity assay using fibroblast cultures revealed that proanthocyanidin is about 120 times less toxic than glutaraldehyde, a currently used tissue stabilizer. In vitro degradation, a criterion often used to examine degree of collagen crosslinking (D. T. Cheung et al., “Mechanism of Crosslinking of Proteins by Glutaraldehyde. IV: In Vitro and in Vivo Stability of a Crosslinked Collagen Matrix,” Connect. Tissue Res. 25: 27-34 (1990)), showed that fixed tissue was resistant to digestion by bacterial collagenase. After subcutaneous implantation for periods ranging from 3 and 6 weeks, no apparent degradation of the glutaraldehyde- or proanthocyanidin-fixed tissue, whereas fixed tissue rapidly disintegrated. More fibroblasts migrate and proliferate inside the proanthocyanidin-fixed implants compared with GA-fixed implants. Tissues crosslinked with proanthocyanidin manifest an enhanced collagen expression and deposition and do not calcify after implantation. Fibroblasts cultured in the presence of proanthocyanidin increased their rate of collagen synthesis. GA, on the other hand, even after thorough rinsing, continues to be cytotoxic, inhibits collagen synthesis, and encourages dystrophic calcification.

The results reported in this Example demonstrate the feasibility of using proanthocyanidin to crosslink collagen in the skin as part of a method for reversing damage to the skin, such as solar damage or incisional trauma. These results demonstrate that proanthocyanidin is an effective crosslinker of collagen that promotes collagen stability and maturation.

### ADVANTAGES OF THE INVENTION

Compositions according to the present invention provide an effective way of promoting the maturation and crosslinking of collagen in the skin of users who apply the compositions. This has the effect of assisting the repair of trauma to the skin such as solar damage and incisional trauma.

Compositions according to the present invention also promote the interaction of proteoglycans, which form part of the ground substance of the skin, with collagen, as well as the stabilization of interactions between such proteoglycans and collagen. These molecules help retain water and aid in giving the skin its natural firmness and elasticity. The promotion of these interactions gives rise to further increased stability of the collagen molecules, assisting the repair of skin damage.

Compositions according to the present invention provide copper to the dermis for essential crosslinking reactions. This promotes the maturation of collagen.

Compositions according to the present invention can be used along with other cosmetics and skin treatments, and can be used in a wide variety of patients and skin types. They are not likely to provoke allergic or inflammatory reactions, and are well tolerated.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.
(ii) about 11.29% of leucine;
(iii) about 14.68% of lysine;
(iv) about 8.48% of phenylalanine;
(v) about 12.43% of threonine;
(vi) about 7.91% of tryptophan;
(vii) about 16.94% of valine;
(viii) about 8.48% of histidine; and
(ix) about 11.29% of arginine

(c) ascorbyl palmitate in a quantity sufficient to accelerate restoration of the integrity and fullness of the skin;

(d) proanthocyanidin in a quantity sufficient to accelerate restoration of the integrity and fullness of the skin;

(e) cupric chloride in a quantity sufficient to accelerate restoration of the integrity and fullness of the skin;

(f) benzyl alcohol in a quantity sufficient to promote delivery of the composition to the skin; and

(g) a topical pharmaceutically acceptable carrier comprising:
(i) water;
(ii) propylene glycol;
(iii) carbopol;
(iv) a surface coated starch polymer;
(v) octyl palmitate;
(vi) isopropyl palmitate;
(vii) silicone fluid;
(viii) a mixture of glyceryl stearate and PEG-100 stearate;
(ix) cetearyl alcohol;
(x) triethanolamine;
(xi) caprylic/capric triglyceride;
(xii) caprylic/capric stearyl triglyceride;
(xiii) natural lavender oils;
(xiv) natural chamomile oils;
(xv) methylparaben;
(xvi) propylparaben; and
(xvii) diazolidinyl urea.

20. A composition for treatment of the skin comprising:

(a) methionine and cysteine in a quantity sufficient to accelerate restoration of the integrity and fullness of the skin;

(b) a mixture of essential amino acids in a quantity sufficient to accelerate restoration of the integrity and fullness of the skin, the mixture of essential amino acids (not including cysteine or methionine) comprising:
(i) about 8.48% of isoleucine;
(ii) about 11.29% of leucine;
(iii) about 14.68% of lysine;
(iv) about 8.48% of phenylalanine;
(v) about 12.43% of threonine;
(vi) about 7.91% of tryptophan;
(vii) about 16.94% of valine;
(viii) about 8.48% of histidine; and
(ix) about 11.29% of arginine

(c) ascorbyl palmitate in a quantity sufficient to accelerate restoration of the integrity and fullness of the skin;

(d) silybin in a quantity sufficient to accelerate restoration of the integrity and fullness of the skin;

(e) cupric chloride in a quantity sufficient to accelerate restoration of the integrity and fullness of the skin;

(f) benzyl alcohol in a quantity sufficient to promote delivery of the composition to the skin; and

(g) a topical pharmaceutically acceptable carrier comprising:
(i) water;
(ii) propylene glycol;
(iii) carbopol;
(iv) a surface coated starch polymer;
(v) octyl palmitate;
(vi) isopropyl palmitate;
(vii) silicone fluid;
(viii) a mixture of glyceryl stearate and PEG-100 stearate;
(ix) cetearyl alcohol;
(x) triethanolamine;
(xi) caprylic/capric triglyceride;
(xii) caprylic/capric stearyl triglyceride;
(xiii) natural lavender oils;
(xiv) natural chamomile oils;
(xv) methylparaben;
(xvi) propylparaben; and
(xvii) diazolidinyl urea.

We claim:

1. A composition for treatment of the skin comprising:
(a) methionine and cysteine in a quantity sufficient to accelerate restoration of the integrity and fullness of the skin;
(b) a mixture of essential amino acids in a quantity sufficient to accelerate restoration of the integrity and fullness of the skin, the mixture of essential amino acids comprising isoleucine, leucine, lysine, phenylalanine, threonine, tryptophan, valine, histidine, and arginine;
(c) at least one antioxidant in a quantity sufficient to accelerate restoration of the integrity and fullness of the skin;
(d) at least one cross-linking agent in a quantity sufficient to accelerate restoration of the integrity and fullness of the skin;
(e) at least one metallic catalyst in a quantity sufficient to accelerate restoration of the integrity and fullness of the skin;
(f) at least one transepidermal delivery agent in a quantity sufficient to promote delivery of the composition to the skin; and
(g) a topical pharmaceutically acceptable carrier.

2. The composition of claim 1 wherein the at least one antioxidant is selected from the group consisting of lipoic acid or a lipoic acid derivative or analogue and ascorbic acid or a derivative of ascorbic acid.

3. The composition of claim 2 wherein the antioxidant is lipoic acid.

4. The composition of claim 2 wherein the antioxidant is ascorbic acid or a derivative of ascorbic acid.

5. The composition of claim 4 wherein the antioxidant is ascorbyl palmitate.

6. The composition of claim 1 wherein the antioxidant is a constituent of ginkgo.

7. The composition of claim 1 wherein the antioxidant is an isoflavone.

8. The composition of claim 1 wherein the metallic catalyst is copper in either its cuprous or cupric ionic form.

9. The composition of claim 1 wherein the mixture of essential amino acids (not including cysteine or methionine) comprises:
(a) from about 5% to about 20% of isoleucine;
(b) from about 5% to about 20% of leucine;
(c) from about 10% to about 25% of lysine;
(d) from about 5% to about 20% of phenylalanine;
(e) from about 5% to about 25% of threonine;
(f) from about 5% to about 20% of tryptophan;
(g) from about 10% to about 25% of valine;
(h) from about 5% to about 20% of histidine; and
(i) from about 5% to about 20% of arginine.

10. The composition of claim 1 wherein the crosslinking agent is a bioflavonoid.

11. The composition of claim 10 wherein the bioflavonoid is selected from the group consisting of quercetin, quercitrin, kaempferol, kaempferol 3-rutinoside, 3′-methoxy kaempferol 3-rutinoside, 5,8,4′-trihydroxyl-6,7-dimethoxyflavone, catechin, epicachetin, epicachetin gallate, epigallocachetin gallate, hesperidin, naringin, rutin, vixetin, proanthocyanidin, apigenin, myricetin, tricetin, quercetin, naringin, kaempferol, luteolin, biflavonyl, silybin, silydianin, and silychristin.

12. The composition of claim 11 wherein the bioflavonoid is proanthocyanidin.

13. The composition of claim 12 wherein the bioflavonoid is silybin.

14. The composition of claim 1 wherein the crosslinking agent is decorin.

15. The composition of claim 1 wherein the transepidermal delivery agent is selected from the group consisting of lower alkyl diols, C₁₀-C₂₀ fatty acids and esters thereof, and C₄-C₂₀ optionally substituted aliphatic alcohols.

16. The composition of claim 15 wherein the transepidermal delivery agent is benzyl alcohol.

17. The composition of claim 1 further comprising a chaotropic agent.

18. The composition of claim 1 wherein the composition further comprises a long-chain fatty acid ester of tocopherol.

19. A composition for treatment of the skin comprising:
(a) methionine and cysteine in a quantity sufficient to accelerate restoration of the integrity and fullness of the skin;
(b) a mixture of essential amino acids in a quantity sufficient to accelerate restoration of the integrity and fullness of the skin, the mixture of essential amino acids (not including cysteine or methionine) comprising:
(i) about 8.48% of isoleucine;
